(19) United States
(12) Patent Application Publication
(10) Pub. No.: US 2026/0272368 A1
(43) Pub. Date: Sep. 17, 2026

(54) METHOD AND APPARATUS FOR PERFORMING ORTHOSTATIC TEST SEQUENCE

(71) Applicant: OURA HEALTH OY, Oulu (FI)

(72) Inventors: Siri SYTELA, Oulu (FI); Heli Koskimäki, Oulu (FI); Heidi SIMILÄ, Oulu (FI)

(21) Appl. No.: 19/565,218

(22) Filed: Mar. 12, 2026

(30) Foreign Application Priority Data
Mar. 12, 2025 (FI) 20255208

(51) Int. Cl. A61B 5/00; A61B 5/0205; A61B 5/024; A61B 5/11; A61B 5/16

(52) U.S. Cl. CPC A61B 5/4035; A61B 5/0205; A61B 5/02405; A61B 5/02427; A61B 5/1116; A61B 5/165; A61B 5/4058; A61B 5/7264; A61B 5/7275; A61B 5/7282; A61B 5/742; A61B 5/6826

(57) ABSTRACT

The present disclosure relates to a method for performing an orthostatic test sequence associated with a user. The method comprises receiving photoplethysmography data associated with the user from at least one wearable device; receiving motion data associated with the user from the at least one wearable device, wherein the motion data coincides with the photoplethysmography data; obtaining user heart rate data based on the photoplethysmography data associated with the user; detecting an ascending user position change based on the motion data associated with the user; identifying a trigger condition for providing a physiological state-related insight associated with the user based on the user heart rate data and the detected ascending user position change; and causing a user device to provide an indication of the physiological state-related insight associated with the user.

### TECHNICAL FIELD

The following relates to devices and data processing, including techniques for performing an orthostatic test sequence.

### BACKGROUND

Some wearable devices may be configured to collect data from users. For example, a wearable device may include one or more sensors that collect physiological data from a user. Some systems associated with the wearable devices may also be able to perform various actions that may provide information, for example, on health conditions or a training response of a user.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter.

According to a first aspect, a method for performing an orthostatic test sequence associated with a user comprises receiving photoplethysmography data associated with the user from at least one wearable device; receiving motion data associated with the user from the at least one wearable device, wherein the motion data coincides with the photoplethysmography data; obtaining user heart rate data based on the photoplethysmography data associated with the user; detecting an ascending user position change based on the motion data associated with the user; identifying a trigger condition for providing a physiological state-related insight associated with the user based on the user heart rate data and the detected position change; and causing a user device to provide an indication of the physiological state-related insight associated with the user.

In an example embodiment of the first aspect, the ascending user position change is from a supine position to a standing position.

In an example embodiment of the first aspect, the method further comprises identifying the trigger condition when the user heart rate data fits a first trend within a pre-determined time period after the user position change.

In an example embodiment of the first aspect, the method further comprises identifying the trigger condition when a difference between a first mean value computed from the user heart rate data within a first time period after the user position change and a second mean value computed from the user heart rate data within a second time period before the user position change exceeds a pre-determined threshold value.

In an example embodiment of the first aspect, the physiological state-related insight comprises an orthostatic intolerance-related insight.

In an example embodiment of the first aspect, the physiological state-related insight comprises an overtraining syndrome-related insight.

In an example embodiment of the first aspect, the physiological state-related insight comprises a stress level insight.

In an example embodiment of the first aspect, the physiological state-related insight comprises a neurological disorder-related insight or a cardiovascular disorder-related insight.

In an example embodiment of the first aspect, the method further comprises receiving further photoplethysmography data associated with the user from the at least one wearable device; receiving further motion data associated with the user from the at least one wearable device, wherein the further motion data coincides with the further photoplethysmography data; obtaining further user heart rate data based on the further photoplethysmography data associated with the user; detecting a supine or sitting user position based on the further motion data associated with the user; identifying a further trigger condition for providing a further physiological state-related insight associated with the user based on the further user heart rate data; and causing the user device to provide an indication of the further physiological state-related insight associated with the user.

In an example embodiment of the first aspect, the method further comprises identifying the further trigger condition when at least part of the further user heart rate data fits a second trend.

In an example embodiment of the first aspect, the method further comprises identifying the further trigger condition when at least one heart rate variability feature value computed from the further user heart rate data meets a corresponding predetermined heart rate variability feature threshold value.

In an example embodiment of the first aspect, the method further comprises generating, based on a plurality of orthostatic test sequences associated with the user performed during asymptomatic days, at least one baseline heart rate variability feature value associated with the user corresponding to at least one heart rate variability feature value computed from the further user heart rate data; and identifying the further trigger condition when the at least one heart rate variability feature value is lower than the at least one baseline heart rate variability feature value by at least a predetermined deviation value.

In an example embodiment of the first aspect, the further physiological state-related insight comprises an orthostatic intolerance symptom warning.

In an example embodiment of the first aspect, the method further comprises causing a graphical user interface of the user device to display the physiological state-related insight.

In an example embodiment of the first aspect, the method further comprises initiating the orthostatic test sequence automatically in response to user setting data.

In an example embodiment of the first aspect, the method further comprises generating baseline data associated with the user based on a plurality of orthostatic test sequences associated with the user; detecting at least one deviation from the baseline data based on a subsequent orthostatic test sequence associated with the user; and causing the user device to provide an indication of the at least one deviation.

According to a second aspect, an apparatus for performing an orthostatic test sequence associated with a user comprises a processor; a memory coupled with the processor; and instructions stored in the memory and executable by the processor to cause the apparatus to receive photoplethysmography data associated with the user from at least one wearable device; receive motion data associated with the user from the at least one wearable device, wherein the motion data coincides with the photoplethysmography data; obtain user heart rate data based on the photoplethysmography data associated with the user; detect an ascending user position change based on the motion data associated with the user; identify a trigger condition for providing a physiological state-related insight associated with the user based on the user heart rate data and the detected position change; and cause a user device to provide an indication of the physiological state-related insight associated with the user.

In an example embodiment of the second aspect, the ascending user position change is from a supine position to a standing position.

In an example embodiment of the second aspect, the instructions are further executable by the processor to cause the apparatus to identify the trigger condition when the user heart rate data fits a first trend within a pre-determined time period after the user position change.

In an example embodiment of the second aspect, the instructions are further executable by the processor to cause the apparatus to identify the trigger condition when a difference between a first mean value computed from the user heart rate data within a first time period after the user position change and a second mean value computed from the user heart rate data within a second time period before the user position change exceeds a pre-determined threshold value.

In an example embodiment of the second aspect, the physiological state-related insight comprises an orthostatic intolerance-related insight.

In an example embodiment of the second aspect, the physiological state-related insight comprises an overtraining syndrome-related insight.

In an example embodiment of the second aspect, the physiological state-related insight comprises a stress level insight.

In an example embodiment of the second aspect, the physiological state-related insight comprises a neurological disorder-related insight or a cardiovascular disorder-related insight.

In an example embodiment of the second aspect, the instructions are further executable by the processor to cause the apparatus to receive further photoplethysmography data associated with the user from the at least one wearable device; receive further motion data associated with the user from the at least one wearable device, wherein the further motion data coincides with the further photoplethysmography data; obtain further user heart rate data based on the further photoplethysmography data associated with the user; detect a supine or sitting user position based on the further motion data associated with the user; identify a further trigger condition for providing a further physiological state-related insight associated with the user based on the further user heart rate data; and cause the user device to provide an indication of the further physiological state-related insight associated with the user.

In an example embodiment of the second aspect, the instructions are further executable by the processor to cause the apparatus to identify the further trigger condition when at least part of the further user heart rate data fits a second trend.

In an example embodiment of the second aspect, the instructions are further executable by the processor to cause the apparatus to identify the further trigger condition when at least one heart rate variability feature value computed from the further user heart rate data meets a corresponding predetermined heart rate variability feature threshold value.

In an example embodiment of the second aspect, the instructions are further executable by the processor to cause the apparatus to generate, based on a plurality of orthostatic test sequences associated with the user performed during asymptomatic days, at least one baseline heart rate variability feature value associated with the user corresponding to at least one heart rate variability feature value computed from the further user heart rate data; and identify the further trigger condition when the at least one heart rate variability feature value is lower than the at least one baseline heart rate variability feature value by at least a predetermined deviation value.

In an example embodiment of the second aspect, the further physiological state-related insight comprises an orthostatic intolerance symptom warning.

In an example embodiment of the second aspect, the instructions are further executable by the processor to cause the apparatus to cause a graphical user interface of the user device to display the physiological state-related insight.

In an example embodiment of the second aspect, the instructions are further executable by the processor to cause the apparatus to initiate the orthostatic test sequence automatically in response to user setting data.

In an example embodiment of the second aspect, the instructions are further executable by the processor to cause the apparatus to generate baseline data associated with the user based on a plurality of orthostatic test sequences associated with the user; detect at least one deviation from the baseline data based on a subsequent orthostatic test sequence associated with the user; and cause the user device to provide an indication of the at least one deviation.

According to a third aspect, a non-transitory computer-readable medium stores code, the code comprising instructions executable by a processor to receive photoplethysmography data associated with the user from at least one wearable device; receive motion data associated with the user from the at least one wearable device, wherein the motion data coincides with the photoplethysmography data; obtain user heart rate data based on the photoplethysmography data associated with the user; detect an ascending user position change based on the motion data associated with the user; identify a trigger condition for providing a physiological state-related insight associated with the user based on the user heart rate data and the detected position change; and cause a user device to provide an indication of the physiological state-related insight associated with the user.

According to a fourth aspect, an apparatus for performing an orthostatic test sequence associated with a user comprises means for: receiving photoplethysmography data associated with the user from at least one wearable device; receiving motion data associated with the user from the at least one wearable device, wherein the motion data coincides with the photoplethysmography data; obtaining user heart rate data based on the photoplethysmography data associated with the user; detecting an ascending user position change based on the motion data associated with the user; identifying a trigger condition for providing a physiological state-related insight associated with the user based on the user heart rate data and the detected position change; and causing a user device to provide an indication of the physiological state-related insight associated with the user.

According to some aspects, there is provided the subject matter of the independent claims. Some further aspects are defined in the dependent claims.

Any example embodiment may be combined with one or more other example embodiments. Many of the attendant features will be more readily appreciated as they become better understood by reference to the following detailed description considered in connection with the accompanying drawings.

### BRIEF DESCRIPTION OF THE DRAWINGS

Some example embodiments will now be described with reference to the accompanying drawings. In the drawings:

FIG. 1 illustrates an example of a system that supports techniques for performing an orthostatic teat sequence associated with a user, in accordance with aspects of the present disclosure.

FIG. 2 illustrates an example of a system that supports techniques for performing an orthostatic test sequence associated with a user, in accordance with aspects of the present disclosure.

FIG. 3 illustrates an example of a system that supports techniques for performing an orthostatic test sequence associated with a user, in accordance with aspects of the present disclosure.

FIG. 4 shows a block diagram of an apparatus that supports techniques for performing an orthostatic test sequence associated with a user, in accordance with aspects of the present disclosure.

FIG. 5 shows a block diagram of a wearable application that supports techniques for providing physiological state-related insights associated with a user, in accordance with aspects of the present disclosure.

FIG. 6 shows a diagram of a system including a device that supports techniques for performing an orthostatic test sequence associated with a user, in accordance with aspects of the present disclosure.

FIG. 7 shows a flowchart illustrating a method that supports techniques for performing an orthostatic test sequence associated with a user, in accordance with aspects of the present disclosure.

FIG. 8 shows a flowchart illustrating a method that supports techniques for performing an orthostatic test sequence associated with a user, in accordance with aspects of the present disclosure.

FIG. 9A illustrates user heart rate data according to an example embodiment.

FIG. 9B illustrates user heart rate data according to an example embodiment.

FIG. 9C illustrates user heart rate data according to an example embodiment.

Like references are used to designate like parts in the accompanying drawings.

### DETAILED DESCRIPTION

Reference will now be made in detail to example embodiments, examples of which are illustrated in the accompanying drawings. The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Although the specification may refer to “an”, “one”, or “some” embodiment(s) in several locations of the text, this does not necessarily mean that each such reference is to the same embodiment(s), or that the feature may not apply to other embodiments. Single features of different embodiments may also be combined to provide other embodiments. Further, when a particular feature, structure, or characteristic is described in connection of an embodiment, it is within the knowledge of one skilled in the art to apply such feature, structure, or characteristic in connection with other embodiments whether or not explicitly described. Furthermore, words “comprising” and “including” should be understood as not limiting the described embodiments/examples to consist of only those features that have been mentioned and such embodiments/examples may contain also features/structures that have not been specifically mentioned.

Furthermore, although numerative terminology, such as “first”, “second”, etc., may be used herein to describe various embodiments, elements, or features, it should be understood that these embodiments, elements, or features should not be limited by this numerative terminology. This numerative terminology is used herein only to distinguish one embodiment, element, or feature from another embodiment, element, or feature. For example, a first trend discussed below could be called a second trend, and vice versa, without departing from the teachings of the present disclosure.

Orthostatic testing, involving a transition from different body positions (e.g., from lying or sitting position to an upright or standing position), may offer valuable insights into the autonomic nervous system (ANS) functioning and cardiovascular regulation reflected through complex adjustments in, e.g., measures of heart rate (HR) and heart rate variability (HRV). Thus, orthostatic testing may be used, e.g., for monitoring a balance between training and recovery, and may reveal signs of athlete overtraining syndrome. Also, orthostatic testing may reveal stress levels even when other physiological stress features might not correspond with subjective feeling of stress levels. Orthostatic testing may also give indications of some syndromes or health conditions such as, e.g., orthostatic hypotension, postural orthostatic tachycardia syndrome (POTS), vasovagal syncope, autonomic neuropathy in diabetes patients, and cardiovascular autonomic dysfunction in Parkinson's disease patients.

Wearable devices, such as wearable ring devices, may be used to collect, monitor, and track physiological data associated with a user based on sensor measurements performed by the wearable device. Examples of physiological data that may be collected by a wearable device may include temperature data, heart rate data, photoplethysmography (PPG) data, blood-oxygen saturation data, and the like. The physiological data collected, monitored, and tracked via the wearable device may be used to gain health insights about the user, such as the user's sleeping patterns, activity patterns, and the like. However, the health insights provided by many conventional wearable devices may not be related to orthostatic testing, and do not enable users to take actions with respect to the health insights relating to orthostatic testing.

Accordingly, aspects of the present disclosure are directed to techniques that enable a user device to provide actionable guidance or insights relating to automatic or induced orthostatic testing to enable users to receive guidance either reactively or proactively.

For example, a wearable device may acquire photoplethysmography data associated with the user and motion data associated with the user from the user throughout the day. The photoplethysmography data may be used to obtain user heart rate data. The motion data may be used to obtain information of the user's position.

The user device may detect an ascending (rising) user position change. Then the user device may identify a trigger condition for providing a physiological state-related insight associated with the user based on the user heart rate data and the detected position change. For example, if the user's position remains in the standing position for a pre-determined time period after the detected ascending user position change, the heart rate data may be used for orthostatic testing. In one example, the trigger condition may be identified when the user heart rate data fits the first trend within the pre-determined time period after the ascending user position change.

In one example, the ascending user position change may be from a supine (reclining, lying) position to a standing position. In another example, the ascending user position change may be from a sitting position to a standing position. In another example, the ascending user position change may be from a supine position to an upright position.

In one example, the physiological state-related insight may comprise an orthostatic intolerance-related insight. Orthostatic intolerance may comprise, for example, orthostatic hypotension, postural orthostatic tachycardia syndrome (POTS), or vasovagal syncope. Traditionally, detection and diagnosis of types of orthostatic intolerance syndromes require a medical professional. Some of such syndromes remain highly underdiagnosed while they can significantly affect quality of life, since in addition to primary symptoms such as dizziness, the syndromes may be associated with, for example, poor sleep quality, fatigue, concentration difficulty, syncope, falls, and increased risk of cardiovascular diseases.

In one example, the orthostatic intolerance may be detected from a greater increase in mean heart rate when the position is changed. In another example, the orthostatic intolerance may be detected from an abnormal heart rate trend after a position change. First the heart rate increases significantly after which it drops fast. In another example, the abnormal heart rate trend, which is related to significant orthostatic intolerance symptoms, can be predicted from lower supine heart rate variability, including root mean square of successive differences (RMSSD) and low (LF) and high (HF) frequency components of heart rate variability.

In one example, the physiological state-related insight may comprise an overtraining syndrome-related (athlete overtraining state-related) insight. Such orthostatic testing may reveal overtraining syndrome without knowledge of baseline level physiological data of the user. In one example, the overtraining syndrome may be detected from an increasing heart rate trend without stabilization after a position change. In another example, the overtraining syndrome may be detected by comparing supine heart rate variability features, especially standard deviation of normal-to-normal (SDNN) intervals and low (LF) and high (HF) frequency components of the heart rate variability, with other similar users (for example, the same gender and similar age). Lower supine heart rate variability may indicate possible overtraining syndrome.

In one example, the physiological state-related insight may comprise a stress level insight. Other physiological data such as, e.g., nocturnal heart rate and heart rate variability may not always correspond with subjective feeling of stress levels, while orthostatic testing may reveal such stress levels. In one example, stress levels can be defined from the change in heart rate variability, especially the low frequency (LF) component of the heart rate variability, when the position is changed. A higher increase in the LF component of heart rate variability may indicate higher stress levels.

In one example, the physiological state-related insight comprises a neurological disorder-related insight or a cardiovascular disorder-related insight. Such disorders may comprise, for example, autonomic neuropathy in diabetes patients, Parkinson's disease, pure autonomic failure, cardiac hypertrophy, or cardiovascular autonomic dysfunction. Traditionally, detection and diagnosis of types of neurological and cardiovascular disorders require a medical professional. These disorders may significantly impair the body functions and require medical treatment. In one example, a neurological and/or a cardiovascular disorder can be detected from abnormal changes in heart rate or heart rate variability when the position is changed. For example, in autonomic neuropathy the increase in mean heart rate is significantly lower than normally and heart rate variability, especially standard deviation of normal-to-normal (SDNN) intervals, root mean square of successive differences (RMSSD), and high (HF) frequency component of heart rate variability, may increase even though normally it should be decreased.

In one example, at least one of the photoplethysmography data associated with the user signal data, the environment, the time of day and other external factors that may affect the test result may be monitored before or during the orthostatic test. The results of the monitoring may then be used to decide whether the orthostatic test can be started or used to modify the individual baseline.

The user device may be caused to provide the physiological state-related insight associated with the user. In one example, the user device itself may provide the physiological state-related insight associated with the user, for example, using a graphical user interface of the user device. For example, the user device may instruct the user of increased risk of cardiovascular diseases and/or provide the user with guidance of how to relieve symptoms relating to orthostatic intolerance.

In one example, preceding orthostatic test sequences may have provided physiological state-related insights comprising an orthostatic intolerance-related insight. The wearable device may acquire further photoplethysmography data associated with the user and further motion data associated with the user from the user throughout the day. The further photoplethysmography data may be used to obtain further user heart rate data. The further motion data may be used to obtain information of the user's position. The user device may detect a supine or sitting user position based on the further motion data associated with the user. Then the user device may identify a further trigger condition for providing a further physiological state-related insight associated with the user based on the further user heart rate data. The user device may provide an indication of the further physiological state-related insight associated with the user. In one example, the user device may identify the further trigger condition when at least part of the further user heart rate data fits a second trend. In another example, the user device may identify the further trigger condition when at least one heart rate variability feature value computed from the further user heart rate data meets a corresponding predetermined heart rate variability feature threshold value. In another example, the user device may generate, based on a plurality of orthostatic test sequences associated with the user performed during asymptomatic days, at least one baseline heart rate variability feature value associated with the user corresponding to at least one heart rate variability feature value computed from the further user heart rate data; and identify the further trigger condition when the at least one heart rate variability feature value is lower than the at least one baseline heart rate variability feature value by at least a predetermined deviation value. The at least one heart rate variability feature value may comprise, for example, root mean square of successive differences (RMSSD) or low (LF) or high (HF) frequency component of heart rate variability. In one example, the further physiological state-related insight may comprise an orthostatic intolerance symptom warning. Because the preceding orthostatic test sequences provided the orthostatic intolerance-related insight, the further user heart rate data may predict symptoms related to the orthostatic intolerance.

In one example, age-group specific data (for example, from crowd sourcing) can be used additionally or alternatively by comparing the age-group specific data with the acquired photoplethysmography data associated with the user (during the orthostatic test sequence) and the comparison results can be used to determine the physiological state-related insights. For example, the physiological state-related insight may indicate “normal result when compared with your age group” etc.

One or more of the examples may provide a solution for easily detecting and monitoring the orthostatic intolerance symptoms without visiting the medical care. Further, one or more of the examples may provide an orthostatic test solution for stress monitoring. Further, one or more of the examples may provide a PPG based solution for overtraining detection and monitoring. Further, a PPG based orthostatic test may provide an easy way to independently monitor multiple health aspects at the same time.

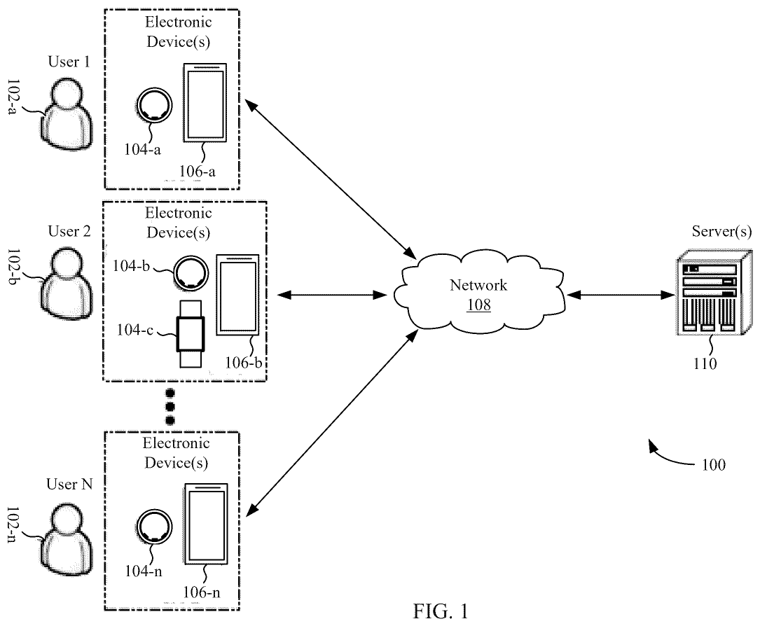

FIG. 1 illustrates an example of a system 100 that supports techniques for performing orthostatic testing and providing physiological state-related insights associated with a user, in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs, auditory signals, haptic signals, visual signals) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, the wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104”, “wearable device 104”, and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for performing an orthostatic test sequence to provide physiological state-related insights associated with a user.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

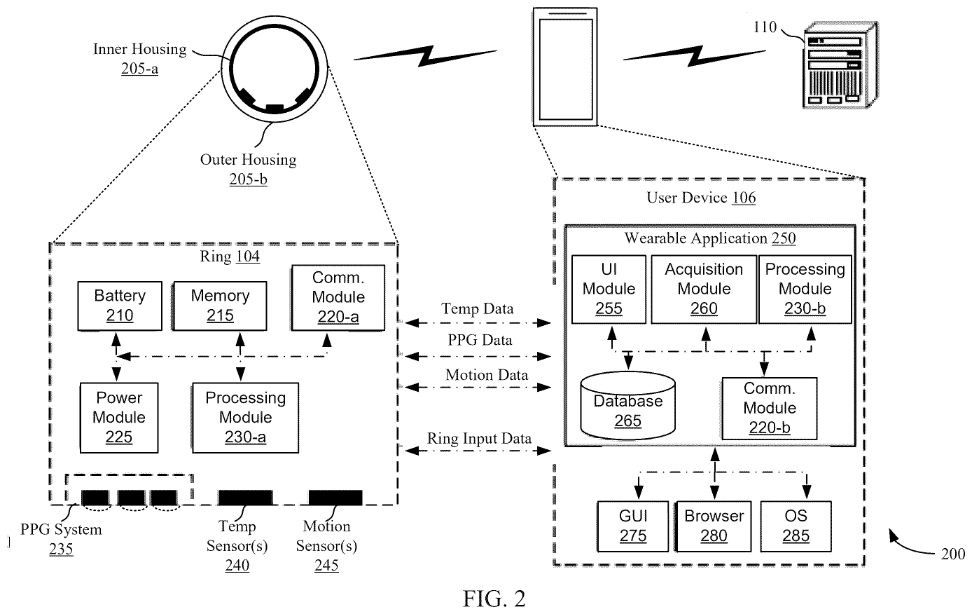

FIG. 2 illustrates an example of a system 200 that supports techniques for providing physiological state related insights associated with a user, in accordance with aspects of the present disclosure. The system 200 may implement, or be implemented by, the system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such as polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electrostatic discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module 220-a) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG signal”) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap occurring after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some implementations, some characteristics can be measured with the wearable device 104 and/or the user device 106, some attributes can be derived (“derived attributes”) from the measurements, and some attributes can be further derived (“further derived attributes”) from the measurements and/or the derived attributes. The measurements may include at least one of the following: photoplethysmography (PPG) data, pulse waveforms, heart rate, motion data. The derived attributes may include at least one of the following: interbeat heart rate, heart rate variability (HRV), metabolic equivalence of task values (METs), orientation values. The further derived attributes may include at least one of the following: velocity, acceleration, changes in position.

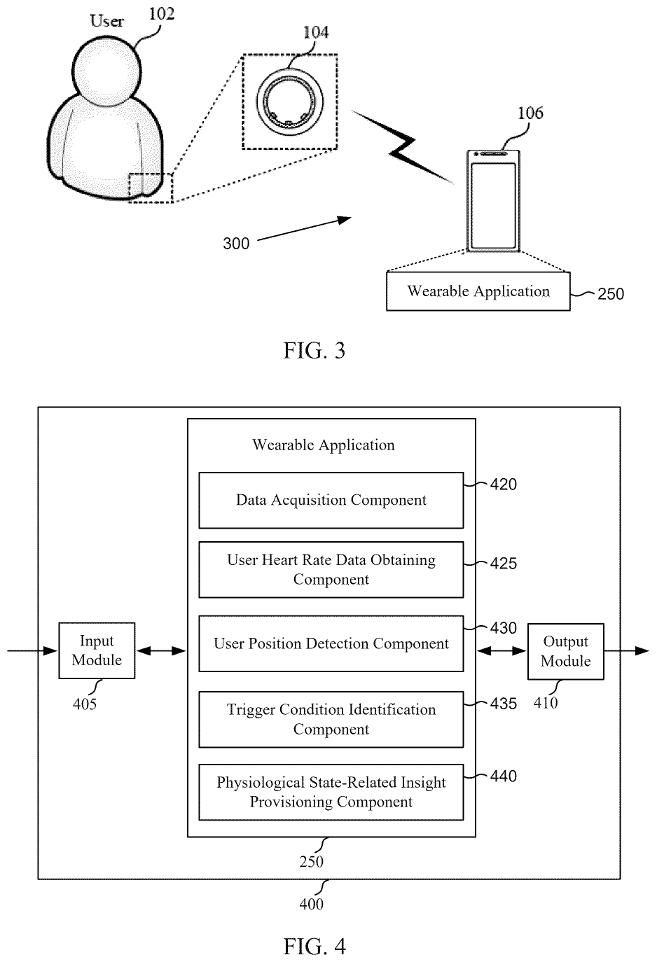

Attendant advantages of the present disclosure may be further shown and described with reference to FIG. 3.

FIG. 3 illustrates an example of a system 300 that supports techniques for performing an orthostatic test sequence associated with a user, in accordance with aspects of the present disclosure. Aspects of the system 300 may implement, or may be implemented by, aspects of the system 100, the system 200, or both. For example, the system 300 may support techniques for performing an orthostatic test sequence associated with a user, as described herein.

The system 300 includes a user 102, a wearable device 104 (e.g., wearable ring device 104), and a user device 106, which may be examples of corresponding devices as described in FIG. 1 and FIG. 2. The user device 106 may execute a wearable application 250.

The system 300 may perform an orthostatic test sequence associated with a user 102. As it is used herein, the term “orthostatic test sequence associated with a user” may refer, for example, to analyzing photoplethysmography data measured while the user position is first, e.g., a supine, reclining, or sitting position and then, e.g., an upright or standing position.

The wearable application 250 may receive photoplethysmography data associated with the user 102 from the wearable device 104. Although FIG. 3 illustrates only one wearable device 104, in other embodiments there may be multiple wearable devices associated with the user 102.

The wearable application 250 may also receive motion data associated with the user 102 coinciding with the photoplethysmography data associated with the user 102 from the wearable device 104. The wearable device 104 may include at least one motion sensor, such as an accelerometer, a gyroscope, a magnetic field sensor, or a combination thereof, to measure the user's movement. The motion sensor is configured to generate motion data that is indicative of the movements of the user. For example, the motion sensor may be configured to determine linear motion information, rotational motion information, orientation information, and the like. Further, such information (linear motion or rotational motion) may be combined or correlated to generate the motion data that is indicative of the user's movement. As mentioned above, the movement data can also be generated by a separate device and retrieved or obtained by the ring or the server.

The wearable application 250 may obtain user heart rate data based on the photoplethysmography data associated with the user 102. The user heart rate data may be determined by the wearable device 104, the wearable application 250, or an external processing device or any combination thereof.

The wearable application 250 may detect an ascending user position change based on the motion data associated with the user 102. For example, the wearable application 250 may detect that the user is first lying down (reclining) for at least some predetermined time period such as, e.g., five or ten minutes, and then stands up and remains standing substantially still for at least another predetermined time period such as, e.g., two or five minutes.

The wearable application 250 may identify a trigger condition for providing a physiological state-related insight associated with the user based on the user heart rate data and the detected position change. As it is used herein, the term “physiological state-related insight associated with the user” may refer, for example, to a message, an instruction, an indication, user guidance information, or user status information representing the user's current physiological status relating to orthostatic intolerance syndromes or symptoms or any combination thereof that may be output by the user device 106. In one example, the wearable application 250 may identify the trigger condition, when the user heart rate data fits a first trend within a pre-determined time period after the detected ascending user position change. In one example, the wearable application 250 may identify the trigger condition, when a difference between a first mean value computed from the user heart rate data within a first time period after the ascending user position change and a second mean value computed from the user heart rate data within a second time period before the ascending user position change exceeds a pre-determined threshold value.

The wearable application 250 may cause a user device to provide an indication of the physiological state-related insight associated with the user.

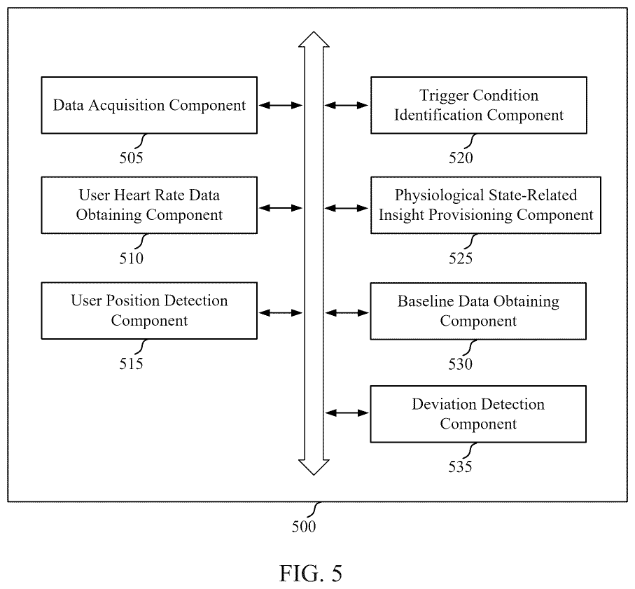

FIG. 4 shows a block diagram of a device 400 that supports techniques for performing an orthostatic test sequence associated with a user, in accordance with aspects of the present disclosure. The device 400 may include an input module 405, an output module 410, and a wearable application 250. The device 400 may also include a processor. Each of these components may be in communication with one another (e.g., via one or more buses).

The input module 405 may provide a means for receiving information such as packets, user data, control information, or any combination thereof associated with various information channels (e.g., control channels, data channels, information channels related to illness detection techniques). Information may be passed on to other components of the device 400. The input module 405 may utilize a single antenna or a set of multiple antennas.

For example, the wearable application 250 may include a data acquisition component 420, a user heart rate data obtaining component 425, a user position detection component 430, a trigger condition identification component 435, and a physiological state-related insight provisioning component 440, or any combination thereof. In some examples, the wearable application 250, or various components thereof, may be configured to perform various operations (e.g., receiving, monitoring, transmitting) using or otherwise in cooperation with the input module 405, the output module 410, or both. For example, the wearable application 250 may receive information from the input module 405, send information to the output module 410, or be integrated in combination with the input module 405, the output module 410, or both to receive information, transmit information, or perform various other operations as described herein.

The data acquisition component 420 may be configured as or otherwise support a means for receiving photoplethysmography data associated with a user from at least one wearable device. The user heart rate data obtaining component 425 may be configured as or otherwise support a means for obtaining user heart rate data associated with the user based on the photoplethysmography data associated with the user. The data acquisition component 420 may be configured as or otherwise support a means for receiving motion data associated with the user from the at least one wearable device, wherein the motion data coincides with the photoplethysmography data, that is, measuring the photoplethysmography data and measuring the motion data from the user have been performed at least in part simultaneously. The user position detection component 430 may be configured as or otherwise support a means for detecting an ascending user position change based on the motion data associated with the user. The trigger condition identification component 435 may be configured as or otherwise support a means for identifying a trigger condition for providing a physiological state-related insight associated with the user based on the user heart rate data and the detected ascending user position change. The physiological state-related insight provisioning component 440 may be configured as or otherwise support a means for causing a user device to provide an indication of the physiological state-related insight associated with the user.

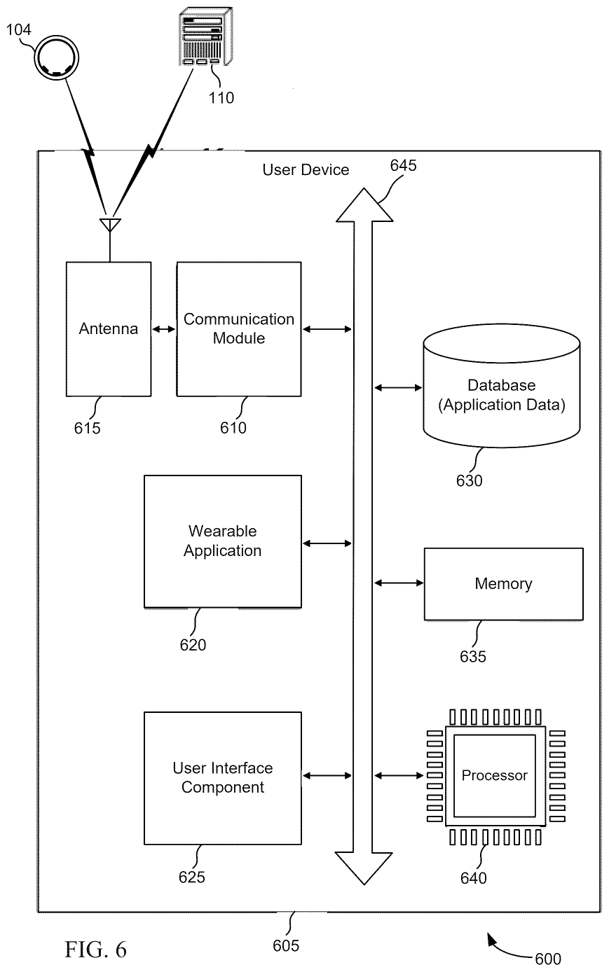

FIG. 5 shows a block diagram of a wearable application 500 that supports techniques for performing an orthostatic test sequence associated with a user, in accordance with aspects of the present disclosure. The wearable application 500 may be an example of aspects of a wearable application or a wearable application 250, or both, as described herein. The wearable application 500, or various components thereof, may be an example of means for performing various aspects of techniques for providing physiological state-related insights associated with a user as described herein. For example, the wearable application 500 may include a data acquisition component 505, a user heart rate data obtaining component 510, a user position detection component 515, a trigger condition identification component 520, a physiological state-related insight provisioning component 525, a physiological baseline obtaining component 530, a deviation detection component 535, or any combination thereof. Each of these components may communicate, directly or indirectly, with one another (e.g., via one or more buses).

The data acquisition component 505 may be configured as or otherwise support a means for receiving photoplethysmography data associated with a user from at least one wearable device. The data acquisition component 505 may be configured as or otherwise support a means for receiving motion data associated with the user from the at least one wearable device. The motion data coincides with the photoplethysmography data. The user heart rate data obtaining component 510 may be configured as or otherwise support a means for obtaining user heart rate data based on the photoplethysmography data associated with the user. The user position detection component 515 may be configured as or otherwise support a means for detecting an ascending user position change based on the motion data associated with the user. The trigger condition identification component 520 may be configured as or otherwise support a means for identifying a trigger condition for providing a physiological state-related insight associated with the user based on the heart rate data and the detected ascending user position change. The physiological state-related insight provisioning component 525 may be configured as or otherwise support a means for causing a user device to provide an indication of the physiological state-related insight associated with the user.

In some examples, the ascending user position change may be from a supine position to a standing position.

In some examples, the trigger condition identification component 520 may be configured as or otherwise support a means for identifying the trigger condition when the user heart rate data fits a first trend within a pre-determined time period after the ascending user position change.

In some examples, the trigger condition identification component 520 may be configured as or otherwise support a means for identifying the trigger condition when a difference between a first mean value computed from the user heart rate data within a first time period after the ascending user position change and a second mean value computed from the user heart rate data within a second time period before the ascending user position change exceeds a pre-determined threshold value.

In some examples, the physiological state-related insight comprises an orthostatic intolerance-related insight.

In some examples, the physiological state-related insight comprises an overtraining syndrome-related insight.

In some examples, the physiological state-related insight comprises a stress level insight.

In some examples, the physiological state-related insight comprises a neurological disorder-related insight or a cardiovascular disorder-related insight. Such disorders may comprise, for example, autonomic neuropathy in diabetes patients, Parkinson's disease, pure autonomic failure, cardiac hypertrophy, or cardiovascular autonomic dysfunction. Traditionally, detection and diagnosis of types of neurological and cardiovascular disorders require a medical professional. These disorders may significantly impair the body functions and require medical treatment.

In some examples, the wearable application 500 may be configured as or otherwise support a means for initiating the orthostatic test sequence automatically in response to user setting data.

In some examples, the data acquisition component 505 may be configured as or otherwise support a means for receiving further photoplethysmography data associated with the user from the at least one wearable device. The data acquisition component 505 may be configured as or otherwise support a means for receiving further motion data associated with the user from the at least one wearable device. The further motion data coincides with the further photoplethysmography data. The user heart rate data obtaining component 510 may be configured as or otherwise support a means for obtaining further user heart rate data based on the further photoplethysmography data associated with the user. The user position detection component 515 may be configured as or otherwise support a means for detecting a supine or sitting user position based on the further motion data associated with the user. The trigger condition identification component 520 may be configured as or otherwise support a means for identifying a further trigger condition for providing a further physiological state-related insight associated with the user based on the further heart rate data. The physiological state-related insight provisioning component 525 may be configured as or otherwise support a means for causing the user device to provide an indication of the further physiological state-related insight associated with the user.

In some examples, the trigger condition identification component 520 may be configured as or otherwise support a means for identifying the further trigger condition when at least part of the further user heart rate data fits a second trend.

In some examples, the trigger condition identification component 520 may be configured as or otherwise support a means for identifying the further trigger condition when at least one heart rate variability feature value computed from the further user heart rate data meets a corresponding predetermined heart rate variability feature threshold value.

In some examples, the physiological baseline obtaining component 530 may be configured as or otherwise support a means for generating, based on a plurality of orthostatic test sequences associated with the user performed during asymptomatic days, at least one baseline heart rate variability feature value associated with the user corresponding to at least one heart rate variability feature value computed from the further user heart rate data. The trigger condition identification component 520 may be configured as or otherwise support a means for identifying the further trigger condition when the at least one heart rate variability feature value is lower than the at least one baseline heart rate variability feature value by at least a predetermined deviation value.

In some examples, the further physiological state-related insight comprises an orthostatic intolerance symptom warning. The orthostatic intolerance symptom warning may be, for example, a warning of dizziness.

In some examples, the physiological state-related insight provisioning component 525 may be configured as or otherwise support a means for causing a graphical user interface of the user device to display the physiological state-related insight.

In some examples, the wearable application 500 may be configured as or otherwise support a means for initiating the orthostatic test sequence automatically in response to user setting data. A user may enable the orthostatic test to be performed automatically (for example, daily) via the wearable application 500. Resting data may be collected, when the user is awake but has not yet gotten out of bed. Then, when the user gets up and remains standing still for a predetermined period of time after standing up, the measurement data collected during this time (i.e., standing still) can be used as data for the orthostatic test.

In some examples, the physiological baseline obtaining component 530 may be configured as or otherwise support a means for obtaining baseline data associated with the user based on a plurality of orthostatic test sequences associated with the user. The deviation detection component 535 may be configured as or otherwise support a means for detecting at least one deviation from the baseline data based on a subsequent orthostatic test sequence associated with the user. For example, when the orthostatic test is done regularly, an individual baseline can be generated for the user and deviations from averages can be monitored. The orthostatic test results may include different HR and HRV values, such as HR rest (average HR when lying down), HRV rest (HRV when lying down), HR peak (one highest heartbeat that occurs after you stand up), HR stand (average HR when standing still), and/or HRV stand (HRV when standing still). The physiological state-related insight provisioning component 525 may be configured as or otherwise support a means for causing the user device to provide an indication of the at least one deviation. As an example, the difference of HR rest, HR peak and HR stand values to averages can be shown to the user.

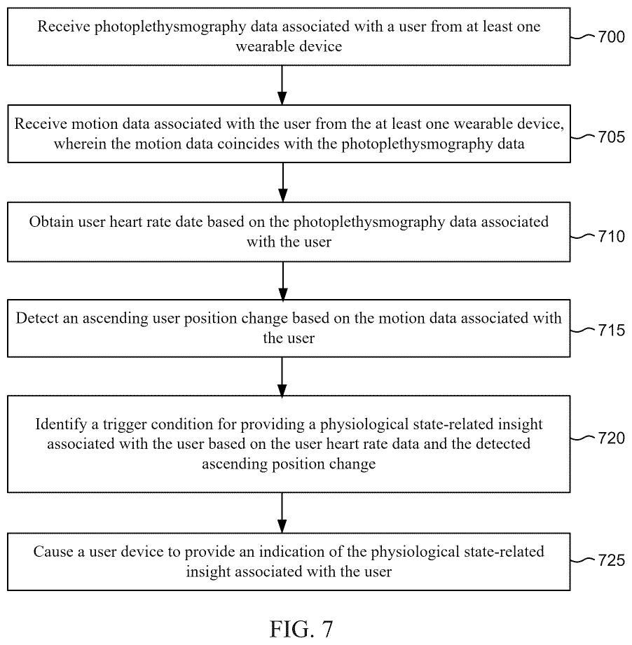

FIG. 6 shows a diagram of a system 600 including a device 605 that supports techniques for performing an orthostatic test sequence associated with a user, in accordance with aspects of the present disclosure. The device 605 may be an example of or include the components of a device 400 as described herein. The device 605 may include an example of a user device 106, as described previously herein. The device 605 may include components for bi-directional communications including components for transmitting and receiving communications with a wearable device 104 and a server 110, such as a wearable application 620, a communication module 610, an antenna 615, a user interface component 625, a database (application data) 630, a memory 635, and a processor 640. These components may be in electronic communication or otherwise coupled (e.g., operatively, communicatively, functionally, electronically, electrically) via one or more buses (e.g., a bus 645).

The communication module 610 may manage input and output signals for the device 605 via the antenna 615. The communication module 610 may include an example of the communication module 220-b of the user device 106 shown and described in FIG. 2. In this regard, the communication module 610 may manage communications with the ring 104 and the server 110, as illustrated in FIG. 2. The communication module 610 may also manage peripherals not integrated into the device 605. In some cases, the communication module 610 may represent a physical connection or port to an external peripheral. In some cases, the communication module 610 may utilize an operating system such as iOS®, ANDROID®, MS-DOS®, MS-WINDOWS®, OS/2®, UNIX®, LINUX®, or another known operating system. In other cases, the communication module 610 may represent or interact with a wearable device (e.g., ring 104), a modem, a keyboard, a mouse, a touchscreen, or a similar device. In some cases, the communication module 610 may be implemented as part of the processor 640. In some examples, a user may interact with the device 605 via the communication module 610, user interface component 625, or via hardware components controlled by the communication module 610.

In some cases, the device 605 may include a single antenna 615. However, in some other cases, the device 605 may have more than one antenna 615, which may be capable of concurrently transmitting or receiving multiple wireless transmissions. The communication module 610 may communicate bi-directionally, via the one or more antennas 615, wired, or wireless links as described herein. For example, the communication module 610 may represent a wireless transceiver and may communicate bi-directionally with another wireless transceiver. The communication module 610 may also include a modem to modulate the packets, to provide the modulated packets to one or more antennas 615 for transmission, and to demodulate packets received from the one or more antennas 615.

The user interface component 625 may manage data storage and processing in a database 630. In some cases, a user may interact with the user interface component 625. In other cases, the user interface component 625 may operate automatically without user interaction. The database 630 may be an example of a single database, a distributed database, multiple distributed databases, a data store, a data lake, or an emergency backup database.

The memory 635 may include RAM and ROM. The memory 635 may store computer-readable, computer-executable software including instructions that, when executed, cause the processor 640 to perform various functions described herein. In some cases, the memory 635 may contain, among other things, a BIOS which may control basic hardware or software operation such as the interaction with peripheral components or devices.

The processor 640 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 640 may be configured to operate a memory array using a memory controller. In other cases, a memory controller may be integrated into the processor 640. The processor 640 may be configured to execute computer-readable instructions stored in a memory 635 to perform various functions (e.g., functions or tasks supporting a method and system for sleep staging algorithms).

For example, the wearable application 620 may be configured as or otherwise support a means for receiving photoplethysmography data associated with a user from at least one wearable device. The wearable application 620 may be configured as or otherwise support a means for receiving motion data associated with a user from the at least one wearable device, wherein the motion data coincides with the photoplethysmography data. The wearable application 620 may be configured as or otherwise support a means for obtaining user heart rate data based on the photoplethysmography data associated with the user. The wearable application 620 may be configured as or otherwise support a means for detecting an ascending user position change based on the motion data associated with the user. The wearable application 620 may be configured as or otherwise support a means for identifying a trigger condition for providing a physiological state-related insight associated with the user based on the user heart rate data and the detected position change. The wearable application 620 may be configured as or otherwise support a means for causing a user device to provide the physiological state-related insight associated with the user.

The wearable application 620 may include an application (e.g., “app”), program, software, or other component which is configured to facilitate communications with a ring 104, server 110, other user devices 106, and the like. For example, the wearable application 620 may include an application executable on a user device 106 which is configured to receive data (e.g., physiological data) from a ring 104, perform processing operations on the received data, transmit and receive data with the servers 110, and cause presentation of data to a user 102.

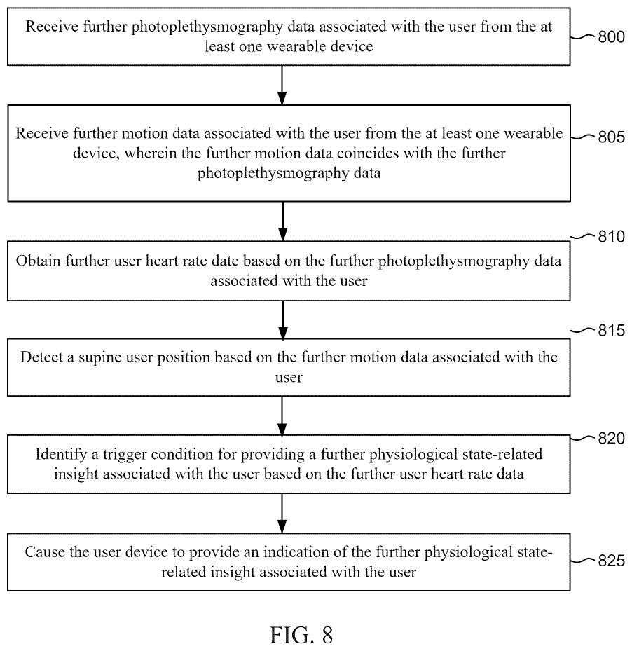

FIG. 7 shows a flowchart illustrating a method that supports techniques for performing an orthostatic test sequence associated with a user, in accordance with aspects of the present disclosure. The operations of the method 700 may be implemented by a user device or its components as described herein. For example, the operations of the method may be performed by a user device as described with reference to FIGS. 1 through 6. In some examples, a user device may execute a set of instructions to control the functional elements of the user device to perform the described functions. Additionally, or alternatively, the user device may perform aspects of the described functions using special-purpose hardware.

At 700, the method may include receiving photoplethysmography data associated with a user from at least one wearable device. The operations of 700 may be performed in accordance with examples as disclosed herein. In some examples, aspects of the operations of 700 may be performed by a data acquisition component 420 as described with reference to FIG. 4.

At 705, the method may include receiving motion data associated with the user from the at least on wearable device, wherein the motion data coincides with the photoplethysmography data. The operations of 705 may be performed in accordance with examples as disclosed herein. In some examples, aspects of the operations of 705 may be performed by a data acquisition component 420 as described with reference to FIG. 4.

At 710, the method may include obtaining user heart rate data based on the photoplethysmography data associated with the user. The operations of 710 may be performed in accordance with examples as disclosed herein. In some examples, aspects of the operations of 710 may be performed by a user heart rate data obtaining component 425 as described with reference to FIG. 4.

At 715, the method may include detecting an ascending user position change based on the motion data associated with the user. The operations of 715 may be performed in accordance with examples as disclosed herein. In some examples, aspects of the operations of 715 may be performed by a user position detection component 430 as described with reference to FIG. 4.

At 720, the method may include identifying a trigger condition for providing a physiological state-related insight associated with the user based on the user heart rate data and the detected ascending user position change. The operations of 720 may be performed in accordance with examples as disclosed herein. In some examples, aspects of the operations of 720 may be performed by a trigger condition identification component 435 as described with reference to FIG. 4.

At 725, the method may include causing a user device to provide the physiological state-related insight associated with the user. The operations of 725 may be performed in accordance with examples as disclosed herein. In some examples, aspects of the operations of 725 may be performed by a physiological state-related insight provisioning component 440 as described with reference to FIG. 4.

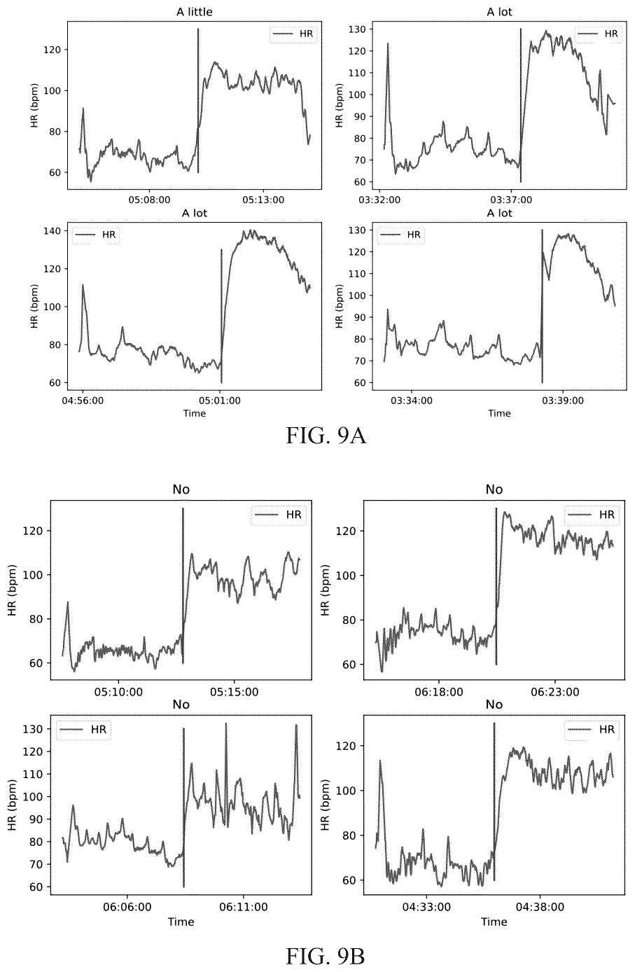

FIG. 8 shows a flowchart illustrating a method that supports techniques for performing a further orthostatic test sequence associated with a user when the physiological state-related insight associated with the user comprises an orthostatic intolerance-related insight, in accordance with aspects of the present disclosure. The operations of the method 800 may be implemented by a user device or its components as described herein. For example, the operations of the method may be performed by a user device as described with reference to FIGS. 1 through 6. In some examples, a user device may execute a set of instructions to control the functional elements of the user device to perform the described functions. Additionally, or alternatively, the user device may perform aspects of the described functions using special-purpose hardware.

At 800, the method may include receiving further photoplethysmography data associated with the user from the at least one wearable device. The operations of 800 may be performed in accordance with examples as disclosed herein. In some examples, aspects of the operations of 800 may be performed by a data acquisition component 420 as described with reference to FIG. 4.

At 805, the method may include receiving further motion data associated with the user from the at least one wearable device, wherein the further motion data coincides with the further photoplethysmography data. The operations of 805 may be performed in accordance with examples as disclosed herein. In some examples, aspects of the operations of 805 may be performed by a data acquisition component 420 as described with reference to FIG. 4.

At 810, the method may include obtaining further user heart rate data based on the further photoplethysmography data associated with the user. The operations of 810 may be performed in accordance with examples as disclosed herein. In some examples, aspects of the operations of 810 may be performed by a user heart rate data obtaining component 425 as described with reference to FIG. 4.

At 815, the method may include detecting a supine or sitting user position based on the further motion data associated with the user. The operations of 815 may be performed in accordance with examples as disclosed herein. In some examples, aspects of the operations of 815 may be performed by a user position detection component 430 as described with reference to FIG. 4.

At 820, the method may include identifying a trigger condition for providing a further physiological state-related insight associated with the user based on the further user heart rate data. The operations of 820 may be performed in accordance with examples as disclosed herein. In some examples, aspects of the operations of 820 may be performed by a trigger condition identification component 435 as described with reference to FIG. 4.

At 825, the method may include causing the user device to provide the further physiological state-related insight associated with the user. The operations of 825 may be performed in accordance with examples as disclosed herein. In some examples, aspects of the operations of 825 may be performed by a physiological state-related insight provisioning component 440 as described with reference to FIG. 4.

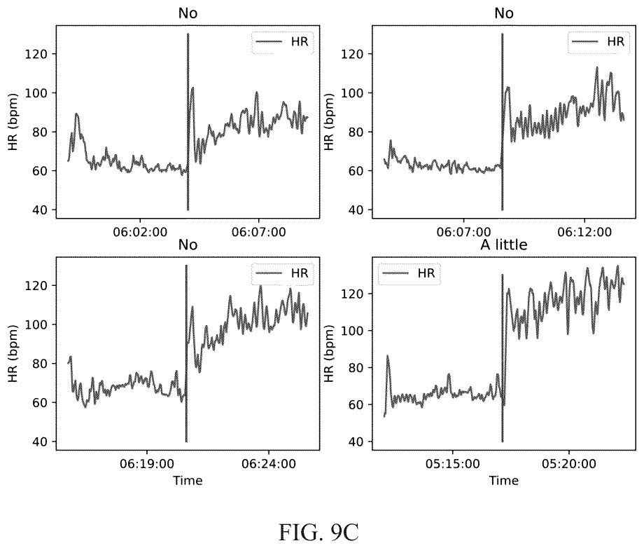

FIG. 9A illustrates examples of user heart rate data of a person with significant orthostatic intolerance during symptomatic days. The vertical straight lines show the detected ascending user position changes. The upper left image shows user heart rate data during a day of a few symptoms and the other three images show user heart rate data during days of a lot of symptoms. After the position change, the heart rate increases significantly high before dropping fast down causing unbearable symptoms like dizziness. This heart rate trend may be used to detect orthostatic intolerance. Before the position change, heart rate variability is lower than during the asymptomatic days in FIG. 9B. Based on the user heart rate variability data it may be possible to warn the user about dizziness that is about to happen before actual symptoms of the dizziness occur.

FIG. 9B illustrates examples of user heart rate data of a person with significant orthostatic intolerance during asymptomatic days. The vertical straight lines show the detected ascending user position changes. After the position change, the heart rate increases and then stabilizes which is a normal behavior and does not lead to symptoms like dizziness. Before the position change, heart rate variability is higher than during the symptomatic days in FIG. 9A.

FIG. 9C illustrates examples of user heart rate data of a person with an athlete overtraining syndrome. After the position change, the heart rate does not stabilize but keeps constantly increasing. Before the position change, heart rate variability is lower than with control users without overtraining syndrome. These features may be used to detect overtraining syndrome when there is no baseline data available from the user before the overtraining state.

Although the subject matter has been described in language specific to structural features and/or acts, it is to be understood that the subject matter defined in the appended claims is not necessarily limited to the specific features or acts described above. Rather, the specific features and acts described above are disclosed as example embodiments of implementing the claims and other equivalent features and acts are intended to be within the scope of the claims.

It will be understood that the benefits and advantages described above may relate to one example embodiment or may relate to several example embodiments. The example embodiments are not limited to those that solve any or all of the stated problems or those that have any or all of the stated benefits and advantages. It will further be understood that reference to ‘an’ item may refer to one or more of those items.

The steps or operations of the methods described herein may be carried out in any suitable order, or simultaneously where appropriate. Additionally, individual steps may be deleted from any of the methods without departing from the scope of the subject matter described herein. Aspects of any of the example embodiments described above may be combined with aspects of any of the other example embodiments described to form further example embodiments without losing the effect sought.

It will be understood that the above description is given by way of example embodiments only and that various modifications may be made by those skilled in the art. The above specification, example embodiments and data provide a complete description of the structure and use of exemplary embodiments. Although various example embodiments have been described above with a certain degree of particularity, or with reference to one or more individual embodiments, those skilled in the art could make numerous alterations to the disclosed example embodiments without departing from scope of this specification.

1. A method for performing an orthostatic test sequence associated with a user, comprising:
receiving photoplethysmography data associated with the user from at least one wearable device;
receiving motion data associated with the user from the at least one wearable device, wherein the motion data coincides with the photoplethysmography data;
obtaining user heart rate data based on the photoplethysmography data associated with the user;
detecting an ascending user position change based on the motion data associated with the user;
identifying a trigger condition for providing a physiological state-related insight associated with the user based on the user heart rate data and the detected ascending user position change; and
causing a user device to provide an indication of the physiological state-related insight associated with the user.

2. A method according to claim 1, wherein the ascending user position change is from a supine position to a standing position.

3. A method according to claim 1, further comprising:
identifying the trigger condition, when the user heart rate data fits a first trend within a pre-determined time period after the user position change.

4. A method according to claim 1, further comprising:
identifying the trigger condition, when a difference between a first mean value computed from the user heart rate data within a first time period after the user position change and a second mean value computed from the user heart rate data within a second time period before the user position change exceeds a pre-determined threshold value.

5. A method according to claim 1, wherein the physiological state-related insight comprises an orthostatic intolerance-related insight.

6. A method according to claim 1, wherein the physiological state-related insight comprises an overtraining syndrome-related insight.

7. A method according to claim 1, wherein the physiological state-related insight comprises a stress level insight.

8. A method according to claim 1, wherein the physiological state-related insight comprises a neurological disorder-related insight or a cardiovascular disorder-related insight.

9. A method according to claim 5, further comprising:
receiving further photoplethysmography data associated with the user from the at least one wearable device;
receiving further motion data associated with the user from the at least one wearable device, wherein the further motion data coincides with the further photoplethysmography data;
obtaining further user heart rate data based on the further photoplethysmography data associated with the user;
detecting a supine or sitting user position based on the further motion data associated with the user;
identifying a further trigger condition for providing a further physiological state-related insight associated with the user based on the further user heart rate data; and
causing the user device to provide an indication of the further physiological state-related insight associated with the user.

10. A method according to claim 9, further comprising:
identifying the further trigger condition when at least part of the further user heart rate data fits a second trend.

11. A method according to claim 9, further comprising:
identifying the further trigger condition when at least one heart rate variability feature value computed from the further user heart rate data meets a corresponding predetermined heart rate variability feature threshold value.

12. A method according to claim 9, further comprising:
generating, based on a plurality of orthostatic test sequences associated with the user performed during asymptomatic days, at least one baseline heart rate variability feature value associated with the user corresponding to at least one heart rate variability feature value computed from the further user heart rate data; and
identifying the further trigger condition when the at least one heart rate variability feature value is lower than the at least one baseline heart rate variability feature value by at least a predetermined deviation value.

13. A method according to claim 9 wherein the further physiological state-related insight comprises an orthostatic intolerance symptom warning.

14. A method according to claim 1, further comprising:
causing a graphical user interface of the user device to display the physiological state-related insight.

15. A method according to claim 1, further comprising:
initiating the orthostatic test sequence automatically in response to user setting data.

16. A method according to claim 1, further comprising:
generating baseline data associated with the user based on a plurality of orthostatic test sequences associated with the user;
detecting at least one deviation from the baseline data based on a subsequent orthostatic test sequence associated with the user; and
causing the user device to provide an indication of the at least one deviation.

17. An apparatus for performing an orthostatic test sequence associated with a user, the apparatus comprising:
a processor;
a memory coupled with the processor; and
instructions stored in the memory and executable by the processor to cause the apparatus to:
receive photoplethysmography data associated with the user from at least one wearable device;
receive motion data associated with the user from the at least one wearable device, wherein the motion data coincides with the photoplethysmography data;
obtain user heart rate data based on the photoplethysmography data associated with the user;
detect an ascending user position change based on the motion data associated with the user;
identify a trigger condition for providing a physiological state-related insight associated with the user based on the user heat rate data and the detected ascending user position change; and
cause a user device to provide an indication of the physiological state-related insight associated with the user.

18. A non-transitory computer-readable medium storing code, the code comprising instructions executable by a processor to:
receive photoplethysmography data associated with the user from at least one wearable device;
receive motion data associated with the user from the at least one wearable device, wherein the motion data coincides with the photoplethysmography data;
obtain user heart rate data based on the photoplethysmography data associated with the user;
detect an ascending user position change based on the motion data associated with the user;
identify a trigger condition for providing a physiological state-related insight associated with the user based on the user heat rate data and the detected ascending user position change; and
cause a user device to provide an indication of the physiological state-related insight associated with the user.
